(19) United States
(12) Patent Application Publication
(10) Pub. No.: US 2008/0097121 A1
(43) Pub. Date: Apr. 24, 2008

(54) Nonionic Liquid Surfactants As Green Solvents

(76) Inventor: Markus M. Hoffmann, Spencerport, NY (US)

Correspondence Address: Dilworth & Barrese, 333 Earle Ovington Blvd, Suite 702, Uniondale, NY, 11553, US

(21) Appl. No.: 11/659,759

(22) PCT Filed: Jul. 29, 2005
(86) PCT No.: PCT/US05/27144

§ 371 Date: Oct. 5, 2007

Related U.S. Application Data
(60) Provisional application No. 60/600,282, filed on Aug. 10, 2004.

(51) Int. Cl. C07C 61/29

(52) U.S. Cl. 562/502

(57) ABSTRACT

A method for preparing a product of a chemical reaction includes conducting the chemical reaction in a solvent medium of one or more liquid nonionic surfactant(s). The liquid nonionic surfactants preferably are CₘEₙ type poly(alkylene oxide) alcohols and are preferably separated from the product without the use of a volatile organic compound.

### CROSS-REFERENCE TO RELATED APPLICATIONS

This application claims priority to U.S. Provisional Application Ser. No. 60/600,282 filed Aug. 10, 2004, the contents of which are incorporated herein by reference.

### BACKGROUND

1. Field of the Invention

The present invention relates to solvents as reaction media, and particularly to the use of nonionic liquid surfactants as reaction media.

2. Background of the Art

Many solvents are known for use as reaction media in which to conduct chemical reactions. Solvents are ubiquitous in chemical synthesis. Only rarely can all reactants be mixed into one homogeneous phase without a solvent. Therefore, a solvent is employed to dissolve all reactants involved. Often, a second different solvent is needed to extract the product from the reaction mixture or to remove leftover reactants or unwanted side products. The chemist has a variety of solvents on hand for these purposes, ranging from very polar to very non-polar solvents. The traditional solvents are usually low-boiling so that they are liquid at room temperature and can easily be removed by evaporation after the reaction is complete. The majority of the traditional solvents are problematic with respect to toxicity, flammability, and environmental compatibility. Because of their low vapor pressures, the risk of exposure through the route of inhalation is inherent for the traditional solvents. Replacement of hazardous solvents is, because of these concerns, one of the principles of “Green Chemistry”.

Efforts are currently being made to minimize the use of volatile organic compounds because of environmental concerns. The expression “volatile organic compound” (VOC) as used herein shall be understood to apply to and designate organic compounds that are characterized by significant vaporization at room temperature (25° C.). VOCs typically have a vapor pressure at room temperature of above 50 mm Hg and a boiling point of less than 260° C. Specific examples of such VOCs include methanol, ethanol, propanol, isopropanol, acetone, ether, light hydrocarbons such as mineral spirits, gasolines, benzenes and other aromatics, halogen substituted alkyl compounds, etc.

Ambient water is one solvent that is considered “green”. However, while water is a great solvent for polar solute and ions, it is a poor solvent for “greasy” non-polar solutes, which forces the synthetic chemist to resort to the more problematic traditional solvents. Besides water, there are two major neoteric “green” solvent systems that currently researchers widely explore as possible replacements for the traditional solvents: supercritical fluids and ionic liquids.

Ionic liquids are also potentially “green” solvents. Ionic liquids are salts that are liquid at ambient conditions. As salts, their vapor pressures are negligibly small, which is their main “green solvent” feature. They are intriguing solvents, and interest in ionic liquids is rising rapidly as is apparent from the explosion of published ionic liquid research articles. As solvents, they can dissolve a wide variety of substances including inorganic salts. Their solvent properties may also be tailored by careful choice of cation-anion combinations and/or by altering functional groups of the organic cation. (The anion is usually inorganic.) Ionic liquids have been explored as solvent media for a wide variety of chemical reactions, including transition metal catalyzed and bio-catalytic reactions. However, ionic liquids are currently very expensive. Moreover, since they are new compounds their toxicity and environmental compatibility is relatively unknown.

Supercritical fluids have been studied and exploited as solvents extensively. These fluids are pressurized and heated to conditions beyond the vapor pressure curve, i.e., the supercritical region of the P-V-T phase surface. Thus, using supercritical fluids as solvents involves working with more expensive pressure-rated equipment, which is a disadvantage. In turn, because the supercritical fluid system is confined, there is inherently the possibility for controlling what is released into the environment. However, the most exciting opportunity that supercritical fluids present as solvent alternatives is that their density along with their solvent characteristics can be changed continuously with pressure and temperature. One might therefore envision to first carry out a reaction in a supercritical fluid under one set of conditions and then change the same supercritical fluid solvent to some other set of conditions that is optimized for separation purposes. Unfortunately, the most green and most commonly used supercritical fluid, supercritical CO₂, is very non-polar and cannot dissolve polar solutes. Even when compressed to high densities, the bulk dielectric constant, ε, barely exceeds a value of 4. In this regard, supercritical water would offer a much larger range of bulk dielectric: from a very polar solvent with ε≈80 to a very non-polar solvent with values of ε below 4. Unfortunately, the critical temperature of about 375° C. and pressures of about 220 bar are very high and demanding on the equipment. Also, water becomes very chemically aggressive under these extreme conditions.

While polyethylene glycol (PEG) has been used as a solvent medium in various reactions, such reactions have typically not involved a process which is entirely “green.” That is, the PEG is often combined with a VOC and/or the product is separated with a VOC.

What is needed is a more convenient solvent medium in which to conduct entirely “green” chemical reactions.

### SUMMARY

A method for preparing a product of a chemical reaction is provided herein. The method comprises conducting the chemical reaction in a solvent medium of one or more liquid nonionic surfactant(s). Preferably, the method also includes separating the surfactant from the product without the use of a volatile organic compound.

The method advantageously employs green chemistry techniques, thereby avoiding the problems associated with the use of VOCs.

### DETAILED DESCRIPTION OF PREFERRED EMBODIMENT(S)

The method of the invention employs one or more liquid nonionic surfactants as the solvent medium in which a chemical reaction is conducted. Thereafter, the surfactant is preferably separated from the reaction product without the use of volatile organic compound(s).

Preferred liquid nonionic surfactants suitable for use as solvent media in the invention include poly(alkylene oxide) nonionic amphiphiles. Particularly preferred solvents include poly(ethylene oxide) alcohols having the general formula CₘH₂ₘ₊₁(OCH₂CH₂)ₙOH, which is commonly shortened to CₘEₙ, wherein preferable values for m can range from about 2 to about 25 and preferable values for n can range from about 1 to about 50. More preferably, m can range from about 5 to about 15 and n can range from about 1 to about 20. Most preferably, m can range from about 8 to about 10 and n can range from about 1 to about 10. The weight average molecular weight Mw of the nonionic surfactant preferably ranges from about 100 to about 2,500 g/mol, more preferably from about 120 to about 1,100 g/mol, and most preferably from about 150 to about 600 g/mol. The vapor pressure of such liquid surfactants is negligible, typically below 10⁻⁹ torr at 25° C. Thus, there is no substantial release of the surfactant into the environment by vaporization. Also, the poly(alkylene oxide) alcohol surfactants of the invention are relatively non-toxic and biodegradable. Moreover, the chemical reaction can be performed in closed containment vessels without creating hazardous high pressure conditions.

Unlike PEG solvent systems, the nonionic surfactants of the invention are amphiphilic. The surfactant molecules typically have a lipophilic portion and a hydrophilic portion. With the CₘEₙ type surfactants the Cₘ portion is lipophilic and non-polar, and the Eₙ portion is hydrophilic and polar. Unlike the CₘEₙ type surfactants, PEG has only the Eₙ portion and is not a surfactant. The CₘEₙ surfactants of the invention are able to dissolve a wide variety of both polar and non-polar substances, thereby maintaining a single phase system for conducting the reaction. That is, reactants do not have to cross a phase boundary and the reaction rate is not limited by transport through a phase boundary.

In the event that reactions are sensitive to the presence of water and that the liquid nonionic surfactant is hygroscopic, it is preferred that water be removed so that the nonionic liquid surfactant is substantially anhydrous when used as the solvent medium.

Any chemical reaction can be performed in accordance with the method of the invention provided that the reactants are soluble or dispersible in the nonionic liquid surfactant and do not chemically react with the nonionic surfactant under the reaction conditions. By way of illustration, suitable reactions include, but are not limited to, Diels-Alder reaction, Heck synthesis, polymerization reactions, nucleophilic substitution reactions, hydrogenation reactions (e.g., hydrogenation of alkynes to cis-olefins) aldol reactions, condensation reactions, Bayliss-Hillman reaction, Suzuki reaction, allylation reaction, deprotection reactions, and the like.

Separation of the surfactant solvent medium from the resulting reaction product is preferably performed without the use of any VOC. In one embodiment, the surfactant is removed by the addition of water, in which the reaction products are typically insoluble. In the event that the surfactant tends to solubilize the reaction product into the water phase by formation of micelles, it may be necessary to keep the concentration of surfactant below the critical micelle formation concentration by adding sufficient water. For water and poly(ethylene oxide) alcohol CₘEₙ type surfactants, that concentration can be about 0.05 mass percent of surfactant to water.

Another type of separation technique which avoids the use of VOC is physical separation, for example by absorption in a chromatographic separation process. For example, CₘEₙ surfactants can be removed from the reaction products by means of liquid chromatography using, for example, silica gel absorbents. Alternative methods of separation include separation by phase separation with water, freezing the surfactant out of solution, separation by extraction with supercritical fluids, stripping with non-reactive compressed gas or supercritical fluid with subsequent rapid decompression such as, for example, the RESS process (Rapid Expansion of Supercritical Solutions).

Features of the invention are illustrated by the examples given below.

### EXAMPLE 1

This example illustrates the performance of a Diels-Alder cyclo-addition reaction in a surfactant reaction medium of the invention.

In a centrifuge tube immersed in a water bath at 55° C., 0.2 grams of maleic anhydride were dissolved in 1.6 mL of a surfactant medium comprising a poly(ethylene oxide) alcohol having the formula CₘEₙ wherein m was a 50/50 mixture of 8 and 10 and n was a distribution averaging 5 to form a single liquid phase mixture. To this mixture was added 0.2 mL of cyclopentadiene freshly distilled from dicyclopentadiene. The reaction mixture was allowed to cool first to room temperature and then to 0° C. in an ice bath. When a few crystals of cis-norbornene-endo-2,3-dicarboxylic anhydride formed on the bottom of the tube, the tube was centrifuged for several minutes and then returned to the ice bath, which accelerated crystal formation.

Obtaining Cis-Norbornene-Endo-2,3-Dicarboxylic Anhydride Product

The cis-norbornene-endo-2,3-dicarboxylic anhydride crystals were collected in a Buchner funnel through vacuum filtration and washed in cold pure water until foaming in the filtrate ceased. The crystals were dried in a desiccator lined with anhydrous calcium chloride (CaCl₂). The overall yield of cis-norbornene-endo-2,3-dicarboxylic anhydride product was 20%.

Obtaining Cis-Norbornene-Endo-2,3-Dicarboxylic Acid Product

For obtaining cis-norbornene-endo-2,3-dicarboxylic acid 5 ml of water was added to the centrifuge tube and quickly mixed to allow the surfactant to dissolve in the water. The tube was centrifuged and the aqueous layer carefully decanted. The separation process was repeated until substantially no surfactant remained in the product as observed by the absence of oily appearance of the water and waxy appearance of the crystals. The crystals were dried in a desiccator lined with calcium chloride. The overall yield of cis-norbornene-endo-2,3-dicarboxylic acid was 89%.

### EXAMPLE 2

The same procedure was followed as in Example 1 except that the value of n of the surfactant was a distribution averaging 2. The yield of cis-norbornene-endo-2,3-dicarboxylic anhydride was 20%. The overall yield of cis-norbornene-endo-2,3-dicarboxylic acid was 45%.

While the above description contains many specifics, these specifics should not be construed as limitations of the invention, but merely as exemplifications of preferred embodiments thereof. Those skilled in the art will envision many other embodiments within the scope and spirit of the invention as defined by the claims appended hereto.

What is claimed is:

1. A method for preparing a product of a chemical reaction, said method comprising conducting the chemical reaction in a solvent medium of one or more liquid nonionic surfactant(s).

2. The method of claim 1 further comprising separating the surfactant from the organic chemical product without the use of a volatile organic compound.

3. The method of claim 1 wherein the nonionic surfactant is a poly(ethylene oxide) alcohol.

4. The method of claim 3 wherein the poly(ethylene oxide) alcohol has the formula CₘH₂ₘ₊₁(OCH₂CH₂)ₙOH wherein m is from 2 to about 25 and n is from 1 to about 50.

5. The method of claim 4 wherein m is from about 5 to about 15 and n is from 1 to about 20.

6. The method of claim 4 wherein m is from about 8 to about 10 and n is from 1 to about 10.

7. The method of claim 4 wherein m is from 8 to 10 and n is a distribution averaging about 5.

8. The method of claim 4 wherein the poly(ethylene oxide) alcohol has a molecular weight of from about 100 to about 2,500 g/mol.

9. The method of claim 4 wherein the poly(ethylene oxide) alcohol has a molecular weight of from about 120 to about 1,100 g/mol.

10. The method of claim 4 wherein the poly(ethylene oxide) alcohol has a molecular weight of from about 150 to about 600 g/mol.

11. The method of claim 2 wherein the chemical reaction is a reaction selected from the group consisting of Diels-Alder reaction, Heck synthesis, polymerization reactions, nucleophilic substitution reactions, hydrogenation reactions, aldol reactions, condensation reactions, Bayliss-Hillman reaction, Suzuki reaction, allylation reaction and deprotection reactions.

12. The method of claim 1 wherein the chemical reaction is a Diels-Alder reaction.

13. The method of claim 2 wherein the product separates out as a water-insoluble solid and the surfactant is separated from the product by means of extraction with water.

14. The method of claim 1 wherein the chemical reaction comprises reacting a conjugated diene with an unsaturated carbonyl compound under Diels-Alder cycloaddition reaction conditions.

15. The method of claim 14 wherein the nonionic surfactant is a poly(ethylene oxide) alcohol having the formula CₘH₂ₘ₊₁(OCH₂CH₂)ₙOH wherein m is from about 2 to about 25 and n is from about 1 to about 50.

16. The method of claim 14 wherein the conjugated diene is cyclopentadiene, the unsaturated carbonyl compound is maleic anhydride, and the nonionic surfactant is a poly(ethylene oxide) alcohol having the formula CₘH₂ₘ₊₁(OCH₂CH₂)ₙOH wherein m is from about 8 to about 10 and n is a distribution averaging about 5.

17. The method of claim 1 wherein separating step (b) is performed chromatographically.

18. A method for making a product of a chemical reaction comprising:
conducting the chemical reaction in a solvent medium of one or more liquid nonionic surfactant(s) having the formula CₘH₂ₘ₊₁(OCH₂CH₂)ₙOH wherein m is from about 2 to about 25 and n is from about 1 to about 50.

19. The method of claim 18 further including the step of separating the surfactant from the product without the use of a volatile organic compound.

20. The method of claim 19 wherein the product separates out from the solvent medium as a water-insoluble solid and the step of separating the surfactant comprises separating the surfactant from the product by means of extraction with water.

21. A reaction mixture comprising:
two or more substances which are chemically reactive with each other under predetermined reaction conditions mixed with a solvent medium of at least one liquid nonionic surfactant having the formula CₘH₂ₘ₊₁(OCH₂CH₂)ₙOH wherein m is from about 2 to about 25 and n is from about 1 to about 50, wherein the reaction mixture is a single liquid phase.
